(19) United States
(12) Patent Application Publication
(10) Pub. No.: US 2017/0253540 A1
(43) Pub. Date: Sep. 7, 2017

(54) METHOD AND APPARATUS FOR PRODUCING HYDROCARBONS

(71) Applicant: Linde Aktiengesellschaft, München (DE)

(72) Inventors: Torben Höfel, München (DE); Helmut Fritz, München (DE)

(21) Appl. No.: 15/510,255

(22) PCT Filed: Sep. 11, 2015
(86) PCT No.: PCT/EP2015/070888

§ 371 Date: Mar. 10, 2017

(30) Foreign Application Priority Data
Sep. 11, 2014 (EP) 14184458.9

(51) Int. Cl. C07C 1/20; C07C 5/25; C07C 4/04

(52) U.S. Cl. CPC C07C 1/20; C07C 4/04; C07C 5/25

(57) ABSTRACT

A method for preparing hydrocarbons is proposed, which comprises, in a catalysis unit (1) using one or more catalysis feed streams (a) containing oxygenates and/or olefins, producing a catalysis product stream (b) containing n-butane, isobutane, 1-butene, 2-butene, isobutene and hydrocarbons with more than four and/or less than four carbon atoms, and which further comprises producing a steam cracking product stream (s) in a steam cracking unit (2) using one or more steam cracking feed streams (g, n, l, r). It is provided that at least the great majority of the hydrocarbons with more than four and/or less than four carbon atoms and the isobutene is eliminated from the catalysis product stream (b) or a part thereof, whereby a stream (g, n) containing at least 5 percent by mole 1-butene and/or 2-butene is formed, and in that this stream (g, n) containing at least 5 percent by mole 1-butene and/or 2-butene or one or more streams (l, r) derived therefrom is or are used as the steam cracking feed stream or streams (g, n, l, r). The invention also relates to a corresponding apparatus (100, 200, 300).

### PRIOR ART

Short-chain olefins such as ethylene and propylene can be produced by steamcracking hydrocarbons. Methods and apparatus for steam cracking hydrocarbons are described for example in the article “Ethylene” in Ullmann's Encyclopedia of Industrial Chemistry, online edition, 15 Apr. 2007, DOI 10.1002/14356007.a10_045.pub2.

Alternative methods of obtaining short-chain olefins are the so-called oxygenate-to-olefin methods (OTO). In oxygenate-to-olefin methods, oxygenates such as methanol or dimethyl ether are introduced into a reaction zone of a reactor in which a catalyst suitable for reacting the oxygenates has been provided. The oxygenates are converted into ethylene and propylene, for example. The catalysts and reaction conditions used in oxygenate-to-olefin methods are basically known to the skilled man.

Oxygenate-to-olefin methods may be carried out with different catalysts. For example, zeolites such as ZSM-5 or SAPO-34 or functionally comparable materials may be used. If ZSM-5 or a comparable material is used, comparatively large amounts of longer-chained (C3plus) hydrocarbons (for designation see below) and comparatively small amounts of shorter-chained (C2minus) hydrocarbons are formed. When SAPO-34 or comparable materials are used, by contrast, shorter-chained (C2minus) hydrocarbons tend to be formed.

Integrated methods and apparatus (combined apparatus) for producing hydrocarbons which comprise steam cracking steps and oxygenate-to-olefin processes or comprise corresponding cracking furnaces and reactors are also known and are described for example in WO 2011/057975 A2 or US 2013/0172627 A1.

Integrated methods of this kind are advantageous, for example, because typically not only the desired short-chain olefins are formed in the oxygenate-to-olefin processes. A substantial proportion of the oxygenates is converted into paraffins and C4plus olefins. At the same time, in steam cracking, not all the furnace feed is cracked into short-chain olefins. In particular, as yet unreacted paraffins may be present in the cracked gas of corresponding cracking furnaces. Moreover, C4plus olefins including diolefins such as butadiene are typically found here. The compounds obtained depend in both cases on the feeds and reaction conditions used.

In the methods proposed in W02011/057975 A2 and US 2013/0172627 A1 the cracked gas of a cracking furnace and the discharge stream from an oxygenate-to-olefin reactor are combined in a joint separating unit and fractionated. After hydrogenation or separation of butadiene, for example, a C4 fraction obtained here may again be subjected to a steam cracking process and/or an oxygenate-to-olefin process. The C4 fraction may be separated into predominantly olefinic and predominantly paraffinic partial fractions.

The present invention is not restricted to oxygenate-to-olefin processes but may theoretically be used with any desired catalytic methods, particularly catalytic methods in which the zeolites described hereinbefore are used as catalysts. Besides methanol and/or dimethyl ether, other oxygenates, for example, other alcohols and/or ethers, may be used as the feed in corresponding catalytic processes.

Similarly, olefinic components such as, for example, a mixture of different C4 hydrocarbons, may be used in corresponding catalytic processes. In this case, the term olefin cracking process (OCP) is used. Different feeds may be introduced into the same reactor or different reactors within the scope of the present invention. For example, an oxygenate-to-olefin process may be carried out in one reactor and an olefin cracking process in another reactor. Both processes, and optionally also a combined process, have the objective, however, of producing a product which is rich in propylene and optionally ethylene from one or more feeds.

The catalytic processes described, which are characterised in that, in particular, the zeolites mentioned are used as catalysts and moreover one or more catalysis feed streams containing oxygenates and/or olefin are used, are thus carried out in a catalysis unit which may contain one or more corresponding reactors.

As already mentioned, the aim of catalytic processes of this kind is to produce products which are rich in propylene and optionally ethylene. Typically, however, in such processes, significant amounts of hydrocarbons with four or more carbon atoms are produced. It is therefore known from the prior art to recycle such hydrocarbons having four or more carbon atoms for catalysis. It is also known to remove such hydrocarbons as product(s). It has also previously been proposed to subject the hydrocarbons having four or more hydrocarbon atoms or parts or fractions thereof to further reaction processes.

U.S. Pat. No. 4,197,185 A discloses a process for producing butane and gasoline of high isooctane content, from a C4 olefin cut issued from a steam cracking unit, comprising the steps of polymerizing at least 90% of the isobutene of the cut mainly to dimers and trimers thereof, hydrogenating the resulting polymerization mixture to normal butane, isooctane and isododecane, supplying the effluent from the hydrogenation unit to a separation zone to recover a gaseous fraction and a liquid mixture, and fractionating the liquid mixture to separate gasoline of high isooctane content, a C3minus fraction and a butane fraction which is recycled to the steam cracking unit.

However, all the known processes have drawbacks. In particular, the efficiency of the utilisation of corresponding hydrocarbons in such processes is often unsatisfactory.

There is therefore a need for improvements to such processes and apparatus for the production of hydrocarbons using the catalytic methods described.

### DISCLOSURE OF THE INVENTION

This problem is solved by a method and an apparatus for producing hydrocarbons having the features of the independent claims. Preferred embodiments are the subject of the dependent claims and the description that follows.

Before the explanation of the features and advantages of the present invention, their basis as well as the terminology used will be explained.

Liquid and gaseous streams may, in the terminology as used herein, be rich in or poor in one or more components, “rich” indicating a content of at least 75%, 90%, 95%, 99%, 99.5%, 99.9% or 99.99% and “poor” indicating a content of at most 25%, 10%, 5%, 1%, 0.1% or 0.01% on a molar, weight or volume basis. The term “predominantly” may correspond to the definition of “rich” provided above, but refers in particular to a content of more than 90%. Liquid and gaseous streams may also, in the terminology as used herein, be enriched or depleted in one or more components, these terms referring to a corresponding content in a starting mixture from which the liquid or gaseous stream was obtained. The liquid or gaseous stream is “enriched” if it contains at least 1.1 times, 1.5 times, 2 times, 5 times, 10 times, 100 times or 1,000 times the amount, “depleted” if it contains at most 0.9 times, 0.5 times, 0.1 times, 0.01 times or 0.001 times the amount of a corresponding component, based on the starting mixture.

A liquid or gaseous stream is “derived” or “formed” from another liquid or gaseous stream (which is also referred to as the starting stream) if it comprises at least some components that were present in the starting stream or are obtained therefrom. A stream derived or formed in this way may be obtained from the starting stream particularly by separating off or deriving a partial stream or one or more components, concentrating or depleting one or more components, chemically or physically reacting one or more components, heating, cooling, pressurising and the like.

Current methods for separating product streams from processes for preparing hydrocarbons include the formation of a number of fractions based on the different boiling points of the components present. In the art, abbreviations are used for these which indicate the carbon number of the hydrocarbons that are predominantly or exclusively present. Thus, a “C1 fraction” is a fraction which predominantly or exclusively contains methane (and by convention also contains hydrogen in some cases, and is then also called a “C1minus fraction”). A “C2 fraction” on the other hand predominantly or exclusively contains ethane, ethylene and/or acetylene. A “C3 fraction” predominantly or exclusively contains propane, propylene, methyl acetylene and/or propadiene. A “C4 fraction” predominantly or exclusively contains butane, butene, butadiene and/or butyne, while the respective isomers may be present in different amounts depending on the source of the C4 fraction. The same also applies to the “C5 fraction” and the higher fractions. Several such fractions may also be combined. For example, a “C2plus fraction” predominantly or exclusively contains hydrocarbons with two or more carbon atoms and a “C2minus fraction” predominantly or exclusively contains hydrocarbons with one or two carbon atoms.

By oxygenates are typically meant ethers and alcohols. Besides methyl tert. butyl ether (MTBE), it is also possible to use, for example, tert. amyl methyl ether (TAME), tert. amyl ethyl ether (TAEE), ethyl tert. butyl ether (ETBE) and diisopropyl ether (DIPE). Alcohols which may be used include for example methanol, ethanol and tert. butanol (TBA, tertiary butyl alcohol). The oxygenates also include, in particular, dimethyl ether (DME, dimethyl ether). The invention is also suitable for use with other oxygenates.

According to a common definition which is also used here, oxygenates are compounds which comprise at least one alkyl group covalently bonded to an oxygen atom. The at least one alkyl group may comprise up to five, up to four or up to three carbon atoms. In particular, the oxygenates which are of interest within the scope of the present invention comprise alkyl groups with one or two carbon atoms, particularly methyl groups. Of particular interest are monohydric alcohols and dialkyl ethers such as methanol and dimethyl ether or corresponding mixtures thereof.

Steam cracking processes are carried out on a commercial scale almost exclusively in tubular reactors in which individual reaction tubes (in the form of coiled tubes, so-called coils) or groups of corresponding reaction tubes can also be operated under different cracking conditions. Reaction tubes or sets of reaction tubes operated under identical or comparable cracking conditions and possibly also tube reactors operated under uniform cracking conditions are also referred to as “cracking furnaces”. A cracking furnace in the terminology used here is thus a construction unit used for steam cracking which subjects a furnace feed to identical or comparable cracking conditions. A steam cracking unit used within the scope of the present invention may comprise one or more cracking furnaces of this kind.

The same also applies, as already mentioned, to the catalysis unit used within the scope of the present invention, in which different reactors can be provided with identical or different catalysts, supplied with identical or different feed streams and operated under identical or different reaction conditions.

The term “steam cracking feed streams” here refers to one or more liquid and/or gaseous streams which are supplied to one or more cracking furnaces. Streams obtained by a corresponding steam cracking process, as described hereinafter, may also be recycled into one or more cracking furnaces and used again as steam cracking feed streams. Suitable steam cracking feed streams include a number of hydrocarbons and hydrocarbon mixtures from ethane to gas oil up to a boiling point of typically 600° C.

A steam cracking feed stream may exclusively comprise so-called “fresh feed”, i.e. a feed which is prepared outside the apparatus and is obtained for example from one or more petroleum fractions, petroleum gas and/or petroleum gas condensates. A steam cracking feed stream may, however, also additionally or exclusively comprise one or more so-called “recycle streams”, i.e. streams that are produced in the apparatus itself and recycled into a corresponding cracking furnace. A steam cracking feed stream may thus also consist of a mixture of one or more fresh feeds with one or more recycle streams.

The steam cracking feed stream is at least partly reacted in the cracking furnace and leaves it as a so-called “crude gas” which can be subjected to after-treatment steps. These encompass, first of all, processing of the crude gas, for example by quenching, cooling and drying, so as to obtain a “cracked gas”. Occasionally the crude gas is also referred to as cracked gas. In the present case, the term “steam cracking product stream” is used for this.

Moreover, the same also applies to the feed stream or streams supplied to one or more catalysis units, which are referred to here as “catalysis feed streams”. The catalysis feed stream or streams are reacted in the catalysis unit in one or more reactors to form one or more product streams referred to here as “catalysis product streams”.

In more recent steam cracking processes and apparatus, mild cracking conditions are increasingly used, as they enable particularly so-called value products such as propylene to be obtained in larger amounts. Basically, processes in which the cracking conditions are adapted to the composition of the steam cracking feed streams are advantageous. Under mild conditions, however, the reaction in the cracking furnace or furnaces is also reduced, so that unreacted compounds are found in comparatively large amounts in the cracking furnace or furnaces and thus lead to a “dilution” of the value products that are to be recovered.

The “cracking conditions” in a cracking furnace mentioned above encompass inter alia the partial pressure of the furnace feed, which may be influenced by the addition of different amounts of steam and the pressure selected in the cracking furnace, the dwell time in the cracking furnace and the temperatures and temperature profiles used therein. The furnace geometry and configuration also play a part.

As the values mentioned influence one another at least partially, the term “cracking severity” has been adopted to characterise the cracking conditions. For liquid furnace feeds, the cracking severity can be described by means of the ratio of propylene to ethylene (P/E) or as the ratio of methane to propylene (M/P) in the cracked gas, based on weight (kg/kg). For gaseous furnace feeds, by contrast, the reaction or conversion of a particular component of the furnace feed can be stated as a measure of the cracking severity. In particular for hydrocarbons with four carbon atoms, the cracking severity can usefully be described by means of the reaction of key components such as n-butane and isobutane. For a technical understanding of the term “cracking severity” reference may be made to the previously mentioned article “Ethylene” in Ullmann's Encyclopedia of Industrial Chemistry.

### ADVANTAGES OF THE INVENTION

The present invention combines the measures described hereinbefore for making optimum use of hydrocarbons as with four or more carbon atoms from a corresponding catalysis process, so as to achieve efficient utilisation with maximum extraction of value and minimal internal recycle streams.

For this purpose the present invention starts from a method for the production of hydrocarbons, which comprises producing a catalysis product stream containing n-butane, isobutane, 1-butene, 2-butene, isobutene and hydrocarbons with more than four and/or less than four carbon atoms, in a catalysis unit using one or more catalysis feed streams containing oxygenates and/or olefins. The catalysis unit comprises, as previously stated, one or more reactors which are supplied with one or more feed streams, referred to here as catalysis feed streams. As explained, the present invention is suitable in oxygenate-to-olefin processes and/or the so-called olefin cracking processes. The reactor or reactors used in a corresponding catalysis unit preferably comprise zeolites as catalysts. As explained, these catalysts may be of the SAPO or ZSM type, in particular. The catalysis unit used within the scope of the present invention is thus set up for a corresponding catalysis process.

The invention further provides that a steam cracking product stream be produced in a steam cracking unit using one or more steam cracking feed streams. The steam cracking process used within the scope of the present invention may be carried out in one or more cracking furnaces, using identical or different steam cracking conditions, as is fundamentally known. For details, see the above explanations. In particular, the steam cracking feed streams used in the steam cracking may be cracked under mild conditions in order to increase the yield of value products. More severe cracking conditions may be used in particular to achieve the highest possible conversion.

According to the invention it is provided that, from the catalysis product stream or part thereof, at least the great majority of the hydrocarbons with more than four and/or less than four carbon atoms and the isobutene are eliminated, whereby a stream containing at least 5 percent by mole, especially at least 10 percent by mole, at least 20 percent by mole or at least 30 percent by mole, and especially at most 90 percent by mole, at most 80 percent by mole, at most 70 percent by mole, at most 60 percent by mole, at most 50 percent by mole or at most 40 percent by mole, 1-butene and/or 2-butene is formed, and that this stream containing 1-butene and/or 2-butene in the amounts mentioned, or one or more streams derived therefrom, is or are used as the steam cracking feed stream or streams. In other words, the invention envisages initially producing a C4 fraction from the catalysis product stream obtained in a corresponding catalysis unit, namely by the above-mentioned removal of the great majority of the hydrocarbons with more than four and/or less than four carbon atoms. The C4 fraction is then also depleted in isobutene, according to the present invention, i.e. in the branched unsaturated C4 hydrocarbon previously present, but not in all unsaturated hydrocarbons. Especially isobutane may also still be present.

Where there is a mention here, and in the following description, of removing the great majority of corresponding hydrocarbons, this may encompass, as mentioned in the introduction, the removal of at least 75% or more of such hydrocarbons. Preferably, corresponding hydrocarbons are removed substantially completely, i.e. in particular by at least 90 or 95%, optionally by at least 99% or more.

Moreover, a C4 fraction of this kind produced by separating off the majority of the hydrocarbons with more than four and/or less than four carbon atoms, contains comparatively few dienes, and is thus “poor” in corresponding dienes in the sense explained above. This is a result of the catalysis process carried out previously, in which correspondingly few dienes, for example butadiene, are formed.

In one or more further steps, within the scope of the present invention and as mentioned, the isobutene is removed. This removal is performed as selectively as possible from the C4 fraction produced. Isobutene should be eliminated as completely as possible or considered neccessary before a corresponding steam cracking process is carried out, as it is comparatively difficult to react in a corresponding steam cracking process and is thus found in a comparatively high concentration in the steam cracking product stream or streams.

The residue remaining, i.e. the stream containing at least 5 percent by mole, especially at least 10 percent by mole, at least 20 percent by mole or at least 30 percent by mole, and especially at most 90 percent by mole, at most 80 percent by mole, at most 70 percent by mole, at most 60 percent by mole, at most 50 percent by mole or at most 40 percent by mole, 1-butene and/or 2-butene, from which other components can also be separated, or one or more streams derived therefrom, is or are supplied in the form of one or more streams to the steam cracking unit used, where it is cracked under identical or different cracking conditions in one or more cracking furnaces. As explained in more detail hereinafter, a corresponding C4 fraction from which butadiene, in particular, can be separated off as product, can be obtained from the steam cracking product stream which is produced in a corresponding steam cracking unit. A C4 fraction of this kind depleted in butadiene can then be recycled at a suitable point, for example combined with the catalysis product stream obtained in the catalysis unit or with a C4 fraction produced therefrom.

The method proposed within the scope of the present invention is particularly efficient as the isobutene which is unsuitable for steam cracking is eliminated here, but all the other components can be fed into the steam cracking largely as a steam cracking feed stream or steam cracking streams. In this way it is also possible to maximise the product quantity of butadiene which is formed in the steam cracking, as, in the absence of isobutene, the cracking conditions can be largely adapted to ensure maximum butadiene production. As unsaturated C4 hydrocarbons other than isobutene are not or not completely removed, this butadiene production is further increased. The present invention therefore provides significant advantages over the prior art where e.g. a full hydrogenation of all unsaturated C4 hydrocarbons is performed, causing an elimination of the isobutene but also of all other unsaturated C4 hydrocarbons. The isobutene eliminated, or a corresponding fraction which contains isobutene or a product produced therefrom, can either be removed as a product or recycled into the catalysis unit.

One essential difference, within the scope of the present invention, from a known method, as disclosed for example in US2013/0172627 A1, is that paraffinic and olefinic components, apart from isobutene, can be fed into the steam cracking and the process results in more than just separation into C4 olefins and C4 paraffins.

Some embodiments of the invention which have already been partly discussed and which are recited in the dependent claims will be further summarised hereinafter.

In particular, it is provided in the process according to the invention that initially the great majority of the hydrocarbons with more than four and/or less than four carbon atoms are separated off, leaving a C4 partial stream, and then the great majority of the isobutene is eliminated from the C4 partial stream. Advantages of corresponding measures have already been explained hereinbefore.

According to a particularly preferred embodiment of the invention it is envisaged that the elimination of the great majority of the isobutene encompasses at least partial reaction of the isobutene and separation of at least some of the reaction product thus formed. Corresponding measures make the elimination of isobutene considerably easier, so that there is no need for complex separation equipment. In particular, the at least partial reaction of the isobutene may suitably be carried out using a catalytic process in which the isobutene is reacted with methanol and/or ethanol to form methyl- or ethyl-tent-butyl ether. Corresponding methods are known in principle from the prior art. Any ethyl- or methyl-tent-butyl ether obtained can easily be separated from a corresponding C4 partial stream and provided as a product. Methyl-tent-butyl ether is particularly suitable as an antiknock agent or as a solvent in the chemical industry. Corresponding products may, however, also be recycled into the catalysis unit and further reacted therein.

Within the scope of a method according to the invention, at least part of a stream remaining after the elimination of the great majority of the isobutene, which comprises the above mentioned amounts of 1-butene and 2-butene, can be used as the, or one of the, steam cracking feed streams if no further separation of components is desired. As such a stream now contains little or virtually no isobutene, the steam cracking process can be flexibly adapted to the particular products that are to be obtained.

As an alternative to the at least partial reaction of the isobutene and the separation of at least some of the reaction product thus formed, it may be provided, in a method according to the invention, that the great majority of the isobutene be separated off by distillation. Separation by distillation has the advantage that isobutene can be obtained directly, i.e. not as a corresponding reaction product.

To make such distillative separation easier, it is particularly advantageous to isomerise the great majority of the 1-butene present in the C4 partial stream to form 2-butene, before the distillative separation of the great majority of the isobutene. The boiling point of isobutene at atmospheric pressure is −6.9° C., while that of 1-butene is −6.47° C. Thus, distillative separation is practically impossible. The boiling point of 2-butene, by contrast, is significantly higher than this, namely 3.7° C. for cis-2-butene and 0.9° C. for trans-2-butene.

It may also be advantageous to form a stream containing predominantly butane and 2-butene from at least part of a stream remaining after the elimination of the great majority of the isobutene and to use this as the steam cracking feed stream or one of the steam cracking feed streams. In other words, after the elimination of the great majority of the isobutene, components other than those mentioned are also eliminated here. The components mentioned, namely n-butane and 2-butene, are particularly suitable as steam cracking feeds.

In addition, it may be advantageous to form a stream containing predominantly isobutane from the components separated off in the formation of a corresponding stream predominantly containing n-butane and 2-butane, and to use this as the steam cracking feed stream or one of the steam cracking feed streams. For example, it may be envisaged within the scope of the present invention to form a stream containing predominantly n-butane and 2-butene from a stream containing isobutene, isobutane and optionally 1-butene as well as n-butane and 2-butene and to feed this into the steam cracking unit. A remaining stream containing isobutene, isobutane and optionally 1-butene may be subjected to a distillation process in which the isobutane is separated off. This may be treated in the steam cracking unit, as mentioned.

As previously mentioned, a corresponding steam cracking product stream contains hydrocarbons with four carbon atoms, including butadiene, as well as hydrocarbons with more than four and/or less than four carbon atoms. A composition of such a stream may be optimised by adjustment of the cracking conditions used, as stated previously.

Advantageously, the great majority of the butadiene and the hydrocarbons with more than four and/or with less than four carbon atoms are separated off from the steam cracking product stream to obtain a residual stream low in butadiene, which predominantly contains hydrocarbons with four carbon atoms. This makes it possible to largely recover the butadiene and to recycle other compounds accordingly. Advantageously, therefore, at least part of the residual stream low in butadiene is combined with at least part of the C4 partial stream mentioned hereinbefore.

An apparatus according to the invention is set up for the manufacture of hydrocarbons. It comprises at least one catalysis unit which is designed to produce a catalysis product stream containing n-butane, isobutane, 1-butene, 2-butene, isobutene and hydrocarbons with more than 4 and/or less than four carbon atoms, using one or more catalysis feed streams containing oxygenates and/or olefins.

Moreover, such an apparatus comprises a steam cracking unit which is designed to generate a steam cracking product stream, using one or more steam cracking feed streams. According to the invention, means are provided in such an apparatus which are designed to eliminate, from the catalysis product stream or a part thereof, at least the great majority of the hydrocarbons with more than four and/or less than four carbon atoms and the isobutene, whereby a stream containing at least 5 percent by mole, especially at least 10 percent by mole, at least 20 percent by mole or at least 30 percent by mole, and especially at most 90 percent by mole, at most 80 percent by mole, at most 70 percent by mole, at most 60 percent by mole, at most 50 percent by mole or at most 40 percent by mole, 1-butene and/or 2-butene is formed. Furthermore, means are provided to use this stream containing 1-butene and/or 2-butene in the amounts mentioned, or one or more streams derived therefrom, as the steam cracking feed stream or streams.

For features and advantages of an apparatus of this kind, reference is specifically made to the foregoing remarks concerning the process according to the invention. In particular, an apparatus of this kind is arranged, by suitable means, to carry out all the steps of a corresponding process.

The invention and preferred embodiments of the invention will be described hereinafter with reference to the appended drawings, which show preferred embodiments of the invention.

### BRIEF DESCRIPTION OF THE DRAWINGS

FIG. 1 shows an apparatus according to one embodiment of the invention, in schematic representation.

FIG. 2 shows an apparatus according to one embodiment of the invention, in schematic representation.

FIG. 3 shows an apparatus according to one embodiment of the invention, in schematic representation.

### EMBODIMENTS OF THE INVENTION

The Figures show corresponding elements with identical reference numerals and are not repeatedly explained, in the interests of clarity. The streams shown in the respective Figures are given identical reference numerals when they have essentially the same or a comparable composition and irrespective of any differences in volume flows. In all the Figures, a catalysis unit is designated 1 and a steam cracking unit is designated 2.

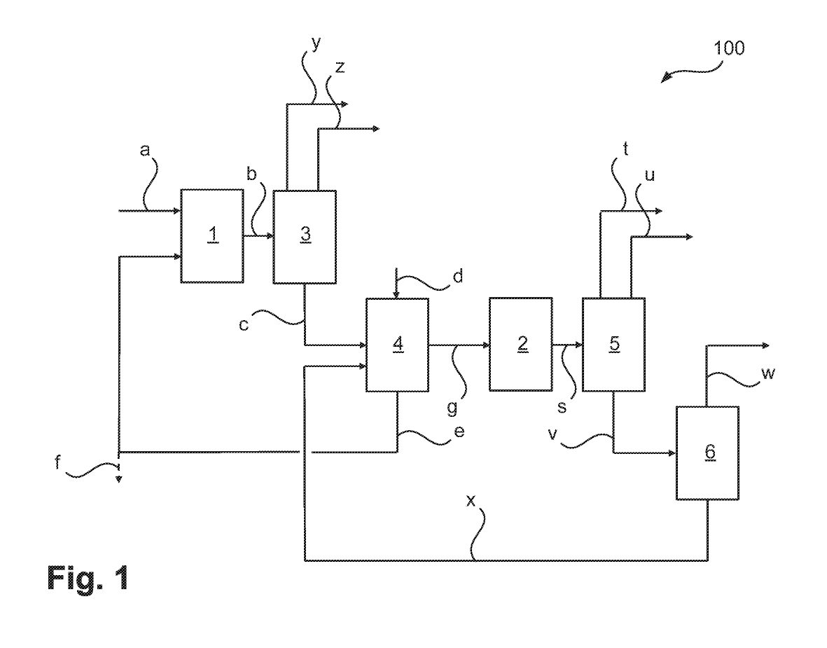

In FIG. 1 an apparatus according to one embodiment of the invention is shown schematically in simplified view and is generally designated 100.

One or more catalysis feed streams, here designated a, containing oxygenates and/or olefins are supplied to the catalysis unit 1. As already mentioned, the catalysis unit 1 may comprise one or more reactors which are operated with a zeolite catalyst. The catalysis unit may additionally be supplied with further streams, in this case the stream e, as explained below.

In the embodiment shown a catalysis product stream b is produced in the catalysis unit 1. It is fed to a separating unit 3, in which a stream c depleted in hydrocarbons with more than four and/or less than four carbon atoms or rich in hydrocarbons with four carbon atoms, i.e. a C4 stream, is obtained from the catalysis product stream b. The streams separated off, here designated y and z, may for example comprise hydrocarbons with five or more or hydrocarbons with three or less carbon atoms, or other such fractions. Streams of this kind may also be processed in a corresponding apparatus and/or recovered as products.

In the embodiment shown the C4 stream c is supplied to a reaction unit 4 in which isobutene contained in the C4 stream c is reacted, for example, with methanol, which is supplied in the form of a stream d, to produce methyl-tent-butyl ether. Methyl-tent-butyl ether or another reaction product with a different compound supplied in the form of the stream d can be drawn off as stream e. As indicated by a dashed arrow, optionally at least some of the corresponding products can be removed from the apparatus as stream f. The remainder of the stream e or the whole of the stream e can be fed into the catalysis unit 1 again and thus used as a recycle stream.

A C4 stream, thus freed from isobutene and now designated g, is fed into the steam cracking unit 2 and processed there in one or more cracking furnaces, optionally also together with further streams which are fed into the same or different cracking furnaces. A steam cracking product stream s is obtained which, as already explained, contains hydrocarbons with four carbon atoms, including butadiene, as well as hydrocarbons with more than four and/or less than four carbon atoms. This stream, here designated s, is fed into a further separating unit 5, in which, initially, by separating off hydrocarbons with more than four and/or less than four carbon atoms, as illustrated here by the streams t and u, a stream v is obtained which predominantly contains hydrocarbons with four carbon atoms, including butadiene. In the embodiment shown the stream v is fed into a butadiene separating unit 6, where the butadiene present is predominantly separated off and discharged from the apparatus as a stream w. A remaining residual stream, here designated x, which is low in butadiene can be combined with the above-mentioned C4 partial stream c and fed into the reaction unit 4.

Depending on the desired result, severe cracking (to maximise ethylene) or mild cracking (to maximise propylene) can be carried out in the steam cracking unit 2. However, irrespective of the cracking severity, there is a tendency for a larger amount of butadiene to be produced, as the feed used in the form of the stream g still contains unsaturated components, namely 1-butene and 2-butene. If an increased amount of butadiene is not wanted, the steam cracking feed stream or streams may be hydrogenated beforehand. In this way, a yield of propylene or ethylene may be increased further. (Partial) hydrogenation and/or hydroisomerisation, which may also be provided, may furthermore be used to hydrogenate any polyunsaturated components still present and to react 1-butene to form 2-butene, which in turn promotes the production of butadiene. All the processes taken together have the advantage that the isobutene which is unsuitable for cracking in the steam cracking unit 2 is eliminated, but all the other components, particularly n-butene, are still available for the cracking.

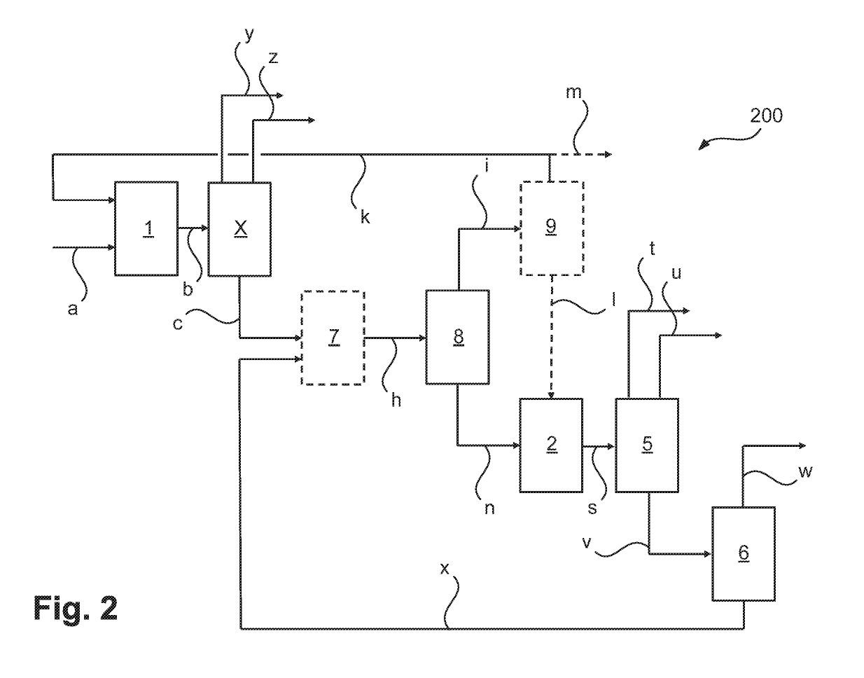

FIG. 2 schematically shows an apparatus according to another embodiment of the invention, generally designated 200.

In contrast to the apparatus 100 shown in FIG. 1, there is no reaction unit 4 provided here for reacting the isobutene, but an isomerisation unit is optionally provided, as illustrated in the form of a block 7 shown by dashed lines. The separating unit designated 3 in FIG. 1 is designated X here but may be of identical configuration. The isomerisation unit 7 is particularly set up for isomerising 1-butene contained in the stream c (and the stream x) to form 2-butene, in order to make subsequent distillation in a distillation unit 8 easier, as explained hereinbefore. The isomerisation unit 7 is particularly set up for hydroisomerisation. By means of the isomerisation in the isomerisation unit 7, preferably exclusively branched C4 components are separated off in the distillation unit 8. In the distillation unit 8 a stream i is obtained which predominantly contains isobutene and isobutane, but not the 1-butene which otherwise also goes over into a corresponding fraction or into the stream i, as this has been isomerised in the isomerisation unit 7. If no such isomerisation is carried out, 1-butene also goes over into the stream i. A further stream obtained in the distillation unit 8, here designated n, essentially contains butane and 2-butene, which are used as steam cracking feed stream and are fed into the steam cracking unit 2.

In the embodiment shown, the stream i is optionally treated in another distillation unit 9, where an isobutene stream k is produced from the stream i, or, if the stream i still contains 1-butene, a stream k containing isobutene and 1-butene is produced, which can be recycled into the catalysis unit. A stream l obtained in the further distillation unit 9 essentially contains isobutane and is supplied as a steam cracking feed stream to the steam cracking unit 2.

If the isomerisation unit 7 and the further distillation unit 9 are available in a corresponding apparatus, essentially all the C4-hydrocarbons of the stream c, apart from isobutene, can be fed into the steam cracking unit 2 and be profitably processed therein. As already mentioned, the isobutene can be recycled into the catalysis unit in the form of the stream k and/or be removed from a corresponding apparatus in the form of the stream m. There is a potential for further maximising the butadiene content in the stream s in the steam cracking unit, as butadiene tends to be formed in larger amounts from 2-butene. Regarding the streams s to x and the units 5 and 6 reference is made to the earlier explanations concerning FIG. 1.

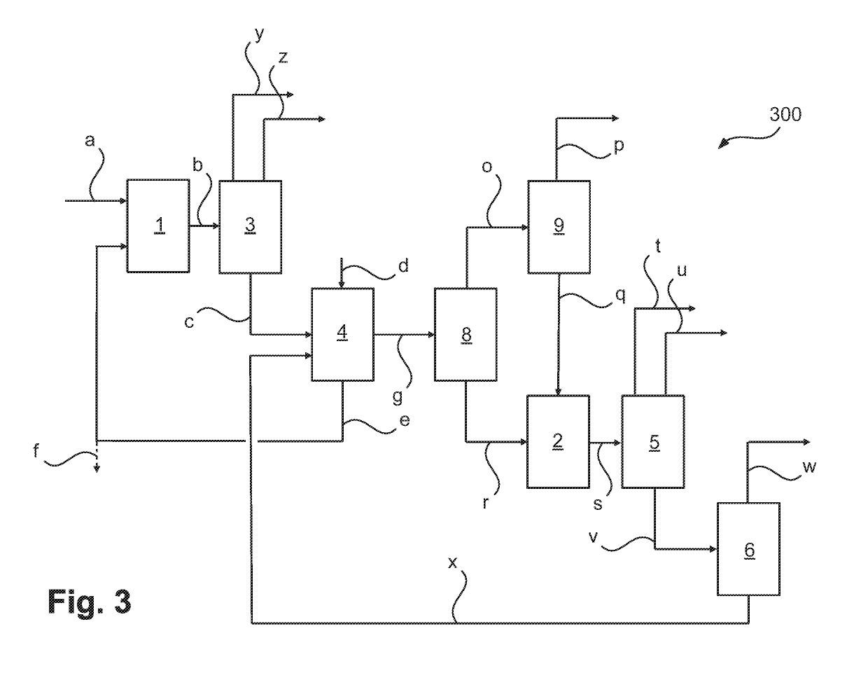

FIG. 3 schematically shows an apparatus according to another embodiment of the invention, generally designated 300.

The apparatus 300 in FIG. 3 corresponds essentially to the apparatus 100 in FIG. 1, up to the reaction unit 4. However, an isobutene-depleted C4 hydrocarbon stream, here also designated g, is fed into a distillation unit which is basically comparable with the distillation unit in the apparatus 200 according to FIG. 2 and is therefore also designated 8 here.

In the distillation unit 8 a partial stream o which essentially contains isobutane and 1-butene is obtained from the isobutene-depleted C4 hydrogen stream g. It is fed into another distillation unit, which is also designated 9 here. In the further distillation unit 9 a stream p essentially containing 1-butene and a stream q essentially containing isobutane are produced from the stream o. The stream q, like a stream r, which is produced in the distillation unit 8 and predominantly contains butane and 2-butene, can be fed into the steam cracking unit 2 as a steam cracking feed stream. For the streams s to x and the units 5 and 6, reference is made to the foregoing explanations of FIG. 1.

Overall, in all the apparatus 100 to 300 shown, hydrocarbon streams with three or less carbon atoms from the separating units 3 or X and 5 can be further processed jointly. Corresponding paraffins separated from these fractions, particularly ethane and propane, can be recycled into the steam cracking unit 2. The same applies to corresponding streams with hydrocarbons having five or more carbon atoms. These may also be further processed jointly. Joint fractionation may be carried out and specific fractions may be recycled into the catalysis unit 1 and/or, optionally after hydrogenation, into the steam cracking unit 2. It should be emphasised that, in certain process variants, other fractions may also be processed, for example fractions that also contain C5, C6 and higher hydrocarbons.

Further embodiments of the present invention might also provide that the isobutene fraction separated off, which also contains 1-butene and isobutene, in particular, should be completely hydrogenated and fed into a steam cracking unit 2, if it is undesirable to feed any recycle streams into the catalysis unit 1. If, for example, recovery of 1-butene is desired, this is also possible by means of a combination of a corresponding reaction unit and subsequent distillation, as shown in FIG. 3. In every case, further feeds such as naphtha and/or ethane or propane may be fed into a steam cracking unit 2.

It should be emphasised that a particular advantage of the present invention arises from the fact that the streams obtained from the catalysis unit b or the streams c formed therefrom contain small amounts of dienes.

1. Method for preparing hydrocarbons, which comprises, in a catalysis unit (1) using one or more catalysis feed streams (a) containing oxygenates and/or olefins, producing a catalysis product stream (b) containing n-butane, isobutane, 1-butene, 2-butene, isobutene and hydrocarbons with more than four and/or less than four carbon atoms, and which further comprises producing a steam cracking product stream (s) in a steam cracking unit (2) using one or more steam cracking feed streams (g, n, l, r), characterised in that at least the great majority of the hydrocarbons with more than four and/or less than four carbon atoms and the isobutene is eliminated from the catalysis product stream (b) or a part thereof, whereby a stream (g, n) containing at least 5 percent by mole 1-butene and/or 2-butene is formed, and in that this stream (g, n) containing at least 5 percent by mole 1-butene and/or 2-butene, or one or more streams (l, r) derived therefrom, is or are used as the steam cracking feed stream or streams (g, n, l, r).

2. Method according to claim 1, wherein initially the great majority of the hydrocarbons with more than four and/or less than four carbon atoms are separated off, leaving a C4 partial stream (c), and subsequently the great majority of the isobutene is eliminated from the C4 partial stream (c).

3. Method according to claim 2, wherein the elimination of the great majority of the isobutene comprises at least partially reacting the isobutene and separating off at least part of the reaction product thus formed.

4. Method according to claim 3, wherein at least part of a stream (g) that remains after the elimination of the great majority of the isobutene is used as the, or one of the, steam cracking feed streams (g).

5. Method according to claim 2, wherein the elimination of the great majority of the isobutene comprises the distillative separation thereof.

6. Method according to claim 5, wherein, before the distillative separation of the great majority of the isobutene, the great majority of the 1-butene contained in the C4 partial stream (c) is isomerised to form 2-butene.

7. Method according to one of claims 2 to 6, wherein a stream (n, r) predominantly containing n-butane and 2-butene is formed from at least part of a stream (g, h) that remains after the elimination of the great majority of the isobutene and is used as the steam cracking feed stream (n, r) or one of the steam cracking feed streams (n, r).

8. Method according to one of claims 2 to 7, wherein a stream (l) is formed which predominantly contains isobutane and is used as the steam cracking feed stream (n, r) or one of the steam cracking feed streams (n, r).

9. Method according to one of claims 2 to 8, wherein the steam cracking product stream (s) contains hydrocarbons with four carbon atoms, including butadiene, as well as hydrocarbons with more than four and/or less than four carbon atoms.

10. Method according to claim 9, wherein the great majority of the butadiene and of the hydrocarbons with more than four and/or less than four carbon atoms are separated from the steam cracking product stream (s), thereby producing a residual stream (x) low in butadiene, which predominantly contains hydrocarbons with four carbon atoms.

11. Method according to one of claims 2 to 10, wherein at least a part of the residual stream low in butadiene is combined with at least a part of the C4 partial stream (c).

12. Apparatus (100, 200, 300) for the production of hydrocarbons, having a catalysis unit (1) which is set up so as to produce, using one or more catalysis feed streams (a) containing oxygenates and/or olefins, a catalysis product stream (b) containing n-butane, isobutane, 1-butene, 2-butene, isobutene and hydrocarbons with more than four and/or less than four carbon atoms, and having a steam cracking unit (2) which is set up to produce a steam cracking product stream (s), using one or more steam cracking feed streams (g, n, l, r), characterised in that means are provided which are set up to eliminate, from the catalysis product stream (b) or a part thereof, at least the great majority of the hydrocarbons with more than four and/or less than four carbon atoms and the isobutene, whereby a stream (g, n) containing at least 5 percent by mole 1-butene and/or 2-butene is formed, and in that means are provided which are set up to use this stream (g, n) containing at least 5 percent by mole 1-butene and/or 2-butene, or one or more streams (l, r) derived therefrom, as the steam cracking feed stream or streams (g, n, l, r).

13. Apparatus (100, 200, 300) according to claim 12, having means which are arranged to carry out a method according to one of claims 1 to 11.
